(19) United States
(12) Patent Application Publication
(10) Pub. No.: US 2022/0317027 A1
(43) Pub. Date: Oct. 6, 2022

(54) APPARATUS FOR BLOOD ANALYSIS

(71) Applicant: ELECTRONICS AND TELECOMMUNICATIONS RESEARCH INSTITUTE, Daejeon (KR)

(72) Inventors: Hyung Ju PARK, Suwon-si (KR); Chul HUH, Daejeon (KR); Bong Kyu KIM, Daejeon (KR); Jeong Won PARK, Seoul (KR)

(21) Appl. No.: 17/507,736

(22) Filed: Oct. 21, 2021

(30) Foreign Application Priority Data
Apr. 5, 2021 (KR) 10-2021-0043826

(51) Int. Cl. G01N 21/31; G01N 33/49

(52) U.S. Cl. CPC G01N 21/31; G01N 33/49; G01N 2201/02

(57) ABSTRACT

Provided is a blood analysis apparatus which may include a capillary tube, a photosensor part on a sidewall of the capillary tube, a cylinder connected to the capillary tube, a pipe body connecting the cylinder and the capillary tube and including a first opening connected to an internal space of the cylinder, a second opening connected to an internal passage of the capillary tube, and a third opening connected to the first opening and the second opening, and a valve arranged on the pipe body and configured to open/close the third opening.

### CROSS-REFERENCE TO RELATED APPLICATION

This U.S. non-provisional patent application claims priority under 35 U.S.C. § 119 of Korean Patent Application No. 10-2021-0043826, filed on Apr. 5, 2021, the entire content of which is hereby incorporated by reference.

### BACKGROUND

### 1. Field of the Invention

The present disclosure herein relates to a blood analysis apparatus, and more particularly, to a blood analysis apparatus capable of measuring blood viscosity.

### 2. Description of Related Art

A health condition, presence/absence of disease, and the like may be detected by analyzing blood. Blood is a non-Newtonian fluid, and a disease may be diagnosed by measuring the viscosity of blood. The viscosity of blood may correlate with particular diseases. It may be presumed that there is a health problem when the viscosity of blood is outside a certain range. In particular, apparatuses capable of measuring the viscosity of blood at various flow rates are required in order to accurately diagnose a disease on the basis of the viscosity of blood. Therefore, blood viscosity analysis equipment used in hospitals has a complicated mechanical structure and large size and includes a large number of components for accurately adjusting the speed of a fluid. As researches about disease diagnosis using the viscosity of blood increase, it is required to carry out researches to develop cheap apparatuses having a simple mechanical structure and capable of measuring the viscosity of blood reliably.

### SUMMARY

The present disclosure provides a blood analysis apparatus capable of measuring the viscosity of blood. The present disclosure also provides a blood analysis apparatus capable of simultaneously obtaining a variety of characteristic information with respect to one blood specimen.

An embodiment of the inventive concept provides a blood analysis apparatus including a capillary tube, a photosensor part on a sidewall of the capillary tube, a cylinder connected to the capillary tube, a pipe body connecting the cylinder and the capillary tube and including a first opening connected to an internal space of the cylinder, a second opening connected to an internal passage of the capillary tube, and a third opening connected to the first opening and the second opening, and a valve arranged on the pipe body and configured to open/close the third opening.

In an embodiment, the third opening may have a diameter equal to an inner diameter of the capillary tube.

In an embodiment, the first opening and the second opening may vertically overlap the internal passage of the capillary tube, and the third opening may be horizontally spaced apart from the internal passage of the capillary tube.

In an embodiment, the third opening may have a diameter of about 0.1 mm to about 10 mm.

In an embodiment, the valve may include a through-hole aligned with the third opening, and may be rotatably coupled to the pipe body.

In an embodiment, the valve may include a through-hole aligned with the third opening, wherein a diameter of the through-hole may be equal to or larger than a diameter of the third opening.

In an embodiment, the photosensor part may include a first photosensor and a second photosensor between the first photosensor and the pipe body, wherein the second photosensor may be configured to transmit and receive light of a wavelength that is longer than that of light transmitted and received by the first photosensor.

In an embodiment, the blood analysis apparatus may further include a piston coupled to the cylinder, wherein the piston may include a first end portion in the cylinder, a second end portion outside the cylinder, and a connecting part penetrating one end of the cylinder and connecting the first end portion and the second end portion.

In an embodiment, the blood analysis apparatus may further include an elastic member between the second end portion and the one end of the cylinder.

In an embodiment, the blood analysis apparatus may further include a piston coupled to one end of the cylinder and a button part on the piston, wherein the button part may include an upper button part and a lower button part coupled to the upper button part, and the button part may be configured to control a maximum movable range of the piston by adjusting a distance between a lower surface of the lower button part and a lower surface of the upper button part.

In an embodiment, a maximum volume of the internal space of the cylinder may be larger than a volume of the internal passage of the capillary tube.

In an embodiment, the first opening may have a diameter equal to an inner diameter of the capillary tube and may be aligned with the internal passage of the capillary tube.

In an embodiment of the inventive concept, a blood analysis apparatus may include: a capillary tube; photosensors on a sidewall of the capillary tube; a pipe structure on one end of the capillary tube; a syringe part on the pipe structure; and a button part on the syringe part, wherein the syringe part includes a cylinder connected to the capillary tube through the pipe structure and a piston coupled to the cylinder, wherein the piston includes a first end portion in the cylinder, a second end portion outside the cylinder, and a connecting part penetrating one end of the cylinder and connecting the first end portion and the second end portion, and wherein the button part is arranged on the second end portion.

In an embodiment, the blood analysis apparatus may further include an elastic member between the one end of the cylinder and the second end portion.

In an embodiment, the blood analysis apparatus may further include a sealing member between the connecting part and the cylinder.

In an embodiment, the pipe structure may include a pipe body including an opening connected to the cylinder and the capillary tube and a valve arranged on the pipe body and configured to open/close the opening.

In an embodiment, the opening may have a diameter equal to an inner diameter of the capillary tube.

In an embodiment, the blood analysis apparatus may further include an absorption sensor arranged adjacent to the one end of the capillary tube.

In an embodiment, the button part may include an upper button part and a lower button part inserted into the upper button part to a first depth, and the button part may be configured to control a maximum movable range of the piston by adjusting the first depth.

In an embodiment, the blood analysis apparatus may further include a control part configured to correct hematocrit using viscosity information and biomarker information about blood.

### BRIEF DESCRIPTION OF THE DRAWINGS

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1 is a cross-sectional view illustrating a blood analysis apparatus according to embodiments of the inventive concept;

FIG. 2 is a perspective view illustrating a pipe structure according to embodiments of the inventive concept;

FIG. 3 is a cross-sectional view illustrating a syringe part according to embodiments of the inventive concept;

FIG. 4, which is a diagram for describing a button part according to embodiments of the inventive concept, is an enlarged cross-sectional view of a part of a blood analysis apparatus;

FIG. 5 is a flowchart illustrating a blood analysis method according to embodiments of the inventive concept;

FIGS. 6A to 8, which are diagrams for describing a blood analysis method according to embodiments of the invention concept, are enlarged cross-sectional views of a part of a blood analysis apparatus;

FIGS. 9 and 10 are front views of a blood analysis apparatus according to embodiments of the inventive concept; and

FIG. 11 is a plan view of a lower sensor part according to embodiments of the inventive concept when viewed from above.

### DETAILED DESCRIPTION

Hereinafter, embodiments of the inventive concept will be described with reference to the accompanying drawings so that the configuration and effects of the inventive concept are sufficiently understood. However, the inventive concept is not limited to the embodiments described below, and may be implemented in various forms and may allow various modifications. Rather, the embodiments are provided so that this disclosure will be thorough and complete, and will fully convey the scope of the inventive concept to those skilled in the art.

Like reference numerals refer to like elements throughout. The embodiments of the inventive concept will be described with reference to front views, perspective views, and/or cross-sectional views that are ideal diagrams of the inventive concept. In the drawings, dimensions of regions are exaggerated for clarity of illustration. Therefore, the regions illustrated in the drawings are merely schematic, and the shapes of the regions exemplify specific shapes of the elements but do not limit the scope of the invention. Various terms are used in various embodiments of the inventive concept to describe various elements, but the elements should not be limited by the terms. Such terms are merely used to distinguish one element from another element. The embodiments described and provided as examples herein include complementary embodiments thereof.

The terminology used herein is not for delimiting the embodiments of the inventive concept but for describing the embodiments of the inventive concept. The terms of a singular form may include plural forms unless otherwise specified. The term “include”, “including”, “comprise” and/or “comprising” used herein does not preclude the presence or addition of one or more other elements.

Hereinafter, embodiments of the inventive concept will be described in detail with reference to the accompanying drawings.

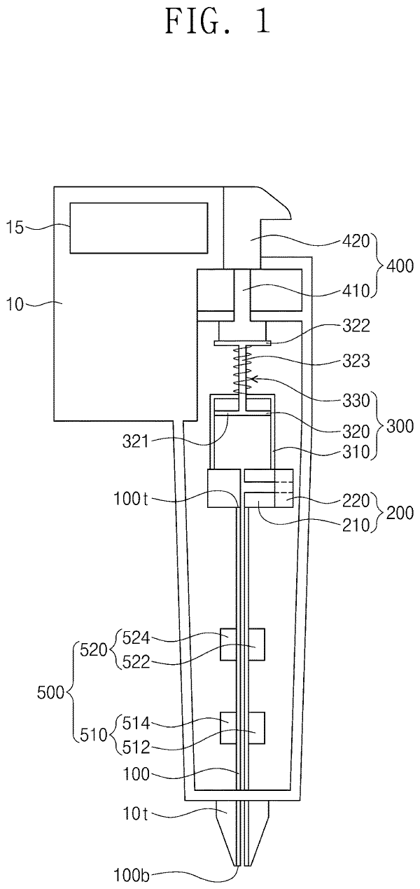

FIG. 1 is a cross-sectional view illustrating a blood analysis apparatus according to embodiments of the inventive concept.

Referring to FIG. 1, the blood analysis apparatus may include a capillary tube 100, a photosensor part 500, a pipe structure 200, a syringe part 300, and a button part 400. The capillary tube 100, the photosensor part 500, the pipe structure 200, the syringe part 300, and the button part 400 may be provided in a housing 10 constituting a body of the blood analysis apparatus.

The capillary tube 100 may extend vertically. The capillary tube 100 may extend from the pipe structure 200 to a tip portion 10t of the housing 10. A top 100t of the capillary tube 100 may contact a lower surface of the pipe structure 200, and a bottom 100b of the capillary tube 100 may be aligned with a sharp end of the tip portion 10t so as to be exposed to a bottom of the blood analysis apparatus. The capillary tube 100 may include a transparent material. The capillary tube 100 may include, for example, at least one of glass or plastic. The capillary tube 100 may include therein a passage through which blood may flow. The passage in the capillary tube 100, for example, may have a circular cross-section. The capillary tube 100, for example, may be a rod-shaped pipe. An inner diameter of the capillary tube 100 may be about 0.1 mm to about 10 mm. A length of the capillary tube 100 may be about 5 cm to about 20 cm. The internal passage of the capillary tube 100 may be surface-treated with a material capable of preventing coagulation of blood. According to embodiments of the inventive concept, a cover layer may be provided on a surface of the internal passage of the capillary tube 100. The cover layer, for example, may include at least one of an ethylenediaminetetraacetic acid (EDTA) or heparin.

The photosensor part 500 may be arranged adjacent to the capillary tube 100. The photosensor part 500 may be located outside the capillary tube 100 and may observe the inside of the capillary tube 100. The photosensor part 500 may be on a sidewall of the capillary tube 100. In detail, the photosensor part 500 may include a first photosensor 510 and a second photosensor 520 arranged on the sidewall of the capillary tube 100. The first photosensor 510 and the second photosensor 520 may be spaced apart from each other vertically. The first photosensor 510 may observe the inside of the capillary tube 100 using light of a shorter wavelength than that of the second photosensor 520. The first photosensor 510 may be arranged closer to the tip portion 10t of the housing 10 than the second photosensor 520. The second photosensor 520 may be arranged between the first photosensor 510 and the pipe structure 200. For example, the first photosensor 510 and the second photosensor 520 may include a photo interrupter.

In detail, the first photosensor 510 may include a first light transmission part 512 and a first light reception part 514. The first light transmission part 512 and the first light reception part 514 may be horizontally spaced apart from each other with the capillary tube 100 therebetween. The second photosensor 520 may include a second light transmission part 522 and a second light reception part 524. The second light transmission part 522 and the second light reception part 524 may be horizontally spaced apart from each other with the capillary tube 100 therebetween. The first light transmission part 512 may emit light of a first wavelength towards the first light reception part 514. The first light reception part 514 may receive the light of the first wavelength that has passed through the capillary tube 100. The second light transmission part 522 may emit light of a second wavelength towards the second light reception part 524. The second light reception part 524 may receive the light of the second wavelength that has passed through the capillary tube 100. The second wavelength may be smaller than the first wavelength. For example, the first wavelength may be about 500 nm to about 600 nm. For example, the second wavelength may be about 800 nm to about 1000 nm.

The pipe structure 200 on one end of the capillary tube 100. The pipe structure 200 may be coupled to the top 100t of the capillary tube 100. The pipe structure 200 may include a pipe body 210 and a valve 220. The pipe body 210 may include therein a passage through which air may flow. The passage of the pipe body 210 may have a 3-channel structure. That is, the pipe body 210 may have three openings connected to each other. The valve 220 may be arranged adjacent to one of the three openings of the pipe body 210. The valve 220 may be configured to open/close one of the three openings of the pipe body 210.

The syringe part 300 may be arranged on the pipe structure 200. The syringe part 300 may be connected to the capillary tube 100 through the pipe structure 200. The syringe part 300 may include a cylinder 310, a piston 320, and an elastic member 330.

The cylinder 310 may have an internal space in which air may flow. A volume of the internal space of the cylinder 310 may be larger than a volume of the passage in the capillary tube 100. One end of the cylinder 310 may be open and connected to the passage in the pipe structure 200. The cylinder 310, for example, may have a cylindrical shape.

The piston 320 and the elastic member 330 may be coupled to another end of the cylinder 310. The piston 320 may be moved vertically so as to adjust a pressure in the internal space of the cylinder 310. The volume of the inner space of the cylinder 310 may be adjusted according to the movement of the piston 320. a maximum volume of the internal space of the cylinder 310 is larger than a volume of the internal passage of the capillary tube 100. the piston 320 may have two end portions 321 and a connecting part 323 connecting the end portions 321 and 322. The first end portion 321 of the piston 320 may be arranged in the cylinder 310. The first end portion 321 of the piston 320 may be moved vertically in a state in which the first end portion 321 is airtightly coupled to an inner wall of the cylinder 310. The second end portion 322 of the piston 320 may be arranged outside the cylinder 310. The elastic member 330 may be provided between the second end portion 322 of the piston 320 and the other end of the cylinder 310. The elastic member 330 may push, in a direction away from the pipe structure 200, the piston 320 pressed towards the pipe structure 200. The elastic member 330, for example, may be a spring.

The button part 400 may be provided on the syringe part 300. The button part 400 may include a lower button part 410 and an upper button part 420. The lower button part 410 may be arranged on the second end portion 322 of the piston 320. The upper button part 420 may be coupled to the lower button part 410. The upper button part 420 may protrude out of the housing 10. The button part 400 may be configured to be vertically moved by external force applied to the upper button part 420.

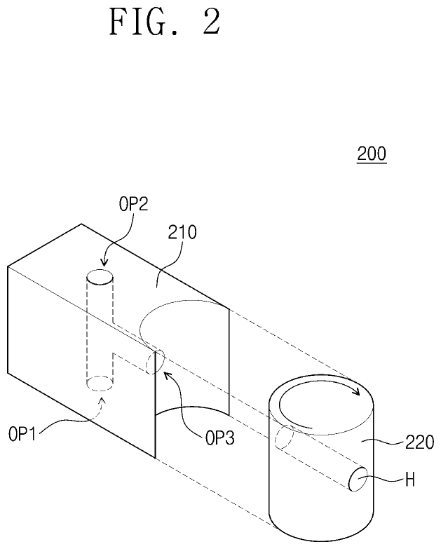

FIG. 2 is a perspective view illustrating a pipe structure according to embodiments of the inventive concept.

Referring to FIGS. 1 and 2, the pipe structure 200 may include a first opening OP1, a second opening OP2, and a third opening OP3. The first opening OP1 may be formed in a lower surface of the pipe body 210 and connected to the capillary tube 100. The first opening OP1 may have a diameter equal to the inner diameter of the capillary tube 100 and may be aligned with the internal passage of the capillary tube 100. The second opening OP2 may be formed in an upper surface of the pipe body 210 and connected to the cylinder 310. The first opening OP1 and the second opening OP2 may overlap each other vertically and may be connected to each other through an internal passage of the pipe body 210. The first opening OP1 and the second opening OP2 may vertically overlap the internal passage of the capillary tube 100.

The third opening OP3 may be provided in a side surface of the pipe body 210. The third opening OP3 may be formed in a direction intersecting a direction in which the first opening OP1 and the second opening OP2 are formed. For example, the third opening OP3 may be formed in a horizontal direction. The third opening OP3 may be formed in a direction to the valve 220. The third opening OP3 may be connected to the first opening OP1 and the second opening OP2 through the internal passage of the pipe body 210. The third opening OP3 may not vertically overlap the first opening OP1 and the second opening OP2. The third opening OP3 may be horizontally spaced apart from the first opening OP1, the second opening OP2, and the internal passage of the capillary tube 100.

According to embodiments of the inventive concept, the valve 220 may be provided on a side surface of the pipe body 210. The valve 220 may be configured to be coupled to the side surface of the pipe body 210 so as to open/close the third opening OP3. The valve 220 may have a cylindrical shape. The side surface of the pipe body 210 facing a side surface of the valve 220 may have a concave shape corresponding to a shape of the valve 220.

In detail, a through-hole H may be formed in the valve 220. The through-hole H may horizontally penetrate the valve 220 so as to form an air passage traversing the valve 220. The through-hole H may be aligned with the third opening OP3 and connected to the passage in the pipe body 210. The through-hole H may inject air into the pipe body 210 or may discharge air in the pipe body 210 to the outside. The valve 220 may be rotatably coupled to the pipe body 210. As the valve 220 rotates, a part of the side surface of the valve 220, in which the through-hole H is not formed, may be airtightly coupled to the side surface of the pipe body 210 so as to seal the third opening OP3.

According to embodiments of the inventive concept, the valve 220 may include one of a ball valve, a gate valve, a globe valve, and a butterfly valve.

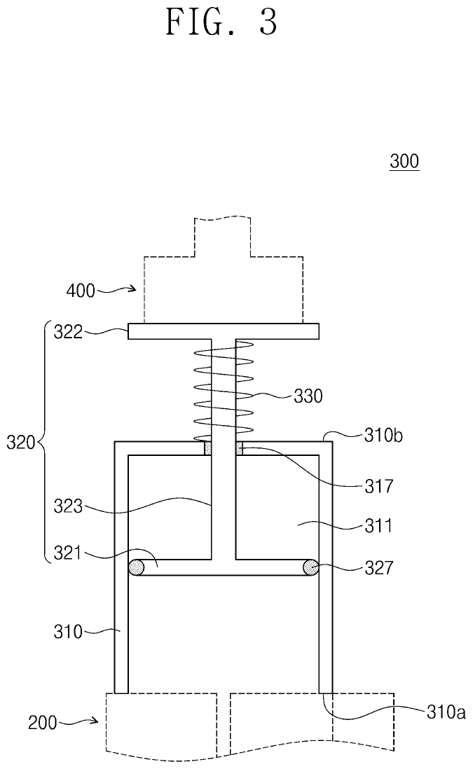

FIG. 3 is a cross-sectional view illustrating a syringe part according to embodiments of the inventive concept.

Referring to FIGS. 1 and 3, the cylinder 310 may have a shape of a half-open tube having one end 310a that is open and another end 310b that is closed. The one end 310a of the cylinder 310 may be connected to the passage in the pipe structure 200. The piston 320 may be coupled to the other end 310b of the cylinder 310. The piston 320 may be pressed by the button part 400 so as to adjust a volume of an internal space 311 of the cylinder 310. The internal space 311 of the cylinder 310 may be defined by a lower surface of the first end portion 321 of the piston 320 and the inner wall of the cylinder 310. For example, when the piston 320 is moved towards the pipe structure 200, the volume of the internal space 311 of the cylinder 310 may reduce. The elastic member 330 may be arranged between the second end portion 322 of the piston 320 and the other end 310b of the cylinder 310. The elastic member 330 may push, in a direction away from the pipe structure 200, the piston 320 moved towards the pipe structure 200.

According to embodiments of the inventive concept, the syringe part 300 may include a piston ring 327 between the first end portion 321 and the inner wall of the cylinder 310. The piston ring 327 may surround a sidewall of the first end portion 321, and may airtightly couple the first end portion 321 with the inner wall of the cylinder 310.

According to embodiments of the inventive concept, the syringe part 300 may include a sealing member 317 between the connecting part 323 and the inner wall of the cylinder 310. The sealing member 317 may surround a sidewall of the connecting part 323.

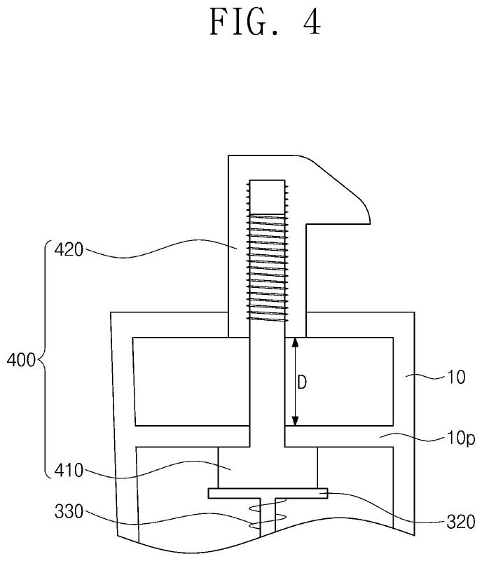

FIG. 4, which is a diagram for describing a button part according to embodiments of the inventive concept, is an enlarged cross-sectional view of a part of a blood analysis apparatus.

Referring to FIGS. 1 and 4, the housing 10 may include an engaging protrusion 10p capable of restricting a movable range of the button part 400. The button part 400 may move upwards until the lower button part 410 contacts a lower surface of the engaging protrusion 10p. The button part 400 may move downwards until the upper button part 420 contacts an upper surface of the engaging protrusion 10p.

The lower button part 410 may be inserted into the upper button part 420. For example, the lower button part 410 may be inserted into the upper button part 420 to a first depth, and the button part 400 may be configured to control a maximum movable range of the piston 320 by adjusting the first depth. According to embodiments of the inventive concept, the upper button part 420 and the lower button part 410 may be screwed to each other. For example, the upper button part 420 may include a female screw part, and the lower button part 410 may include a male screw part. The upper button part 420 and the lower button part 410 may have beveled threads. A length of the button part 400 may be adjusted by rotating one of the upper button part 420 and the lower button part 410. As the length of the button part 400 is adjusted, a distance D between a lower surface of the upper button part 420 and the upper surface of the engaging protrusion 10p may be adjusted. Accordingly, a maximum movable range of the button part 400 may be controlled.

Referring back to FIG. 1, a control part 15 may be provided on the housing 10. The control part 15 may be connected to the pipe structure 200, the syringe part 300, the button part 400, and the photosensor part 500. The control part 15 may include hardware and/or software. The hardware of the control part 15 may include, for example, a processor and a memory. The processor may control overall operation of the pipe structure 200, the syringe part 300, the button part 400, and the photosensor part 500 and a flow of signals between components of the blood analysis apparatus, and may process data. The memory may store signals and data processed by the processor. The software of the control part 15, for example, may be implemented with programming or scripting language including various algorithms implemented with a combination of data structure, processes, routines, or other programming components. According to embodiments of the inventive concept, the control part 15 may be configured to correct hematocrit using blood viscosity information and biomarker information. The control part 15 may implement an algorithm for correcting hematocrit measured on the basis of blood viscosity and biomarker information. A method for correcting hematocrit in the control unit 15 will be described later.

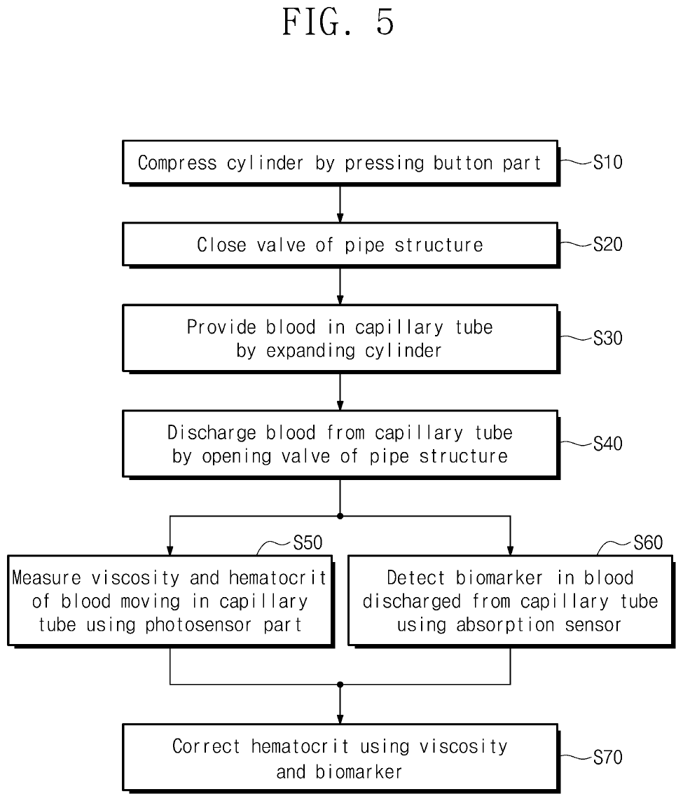

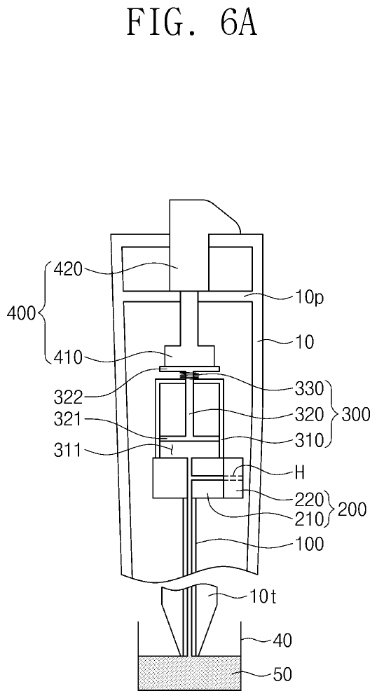

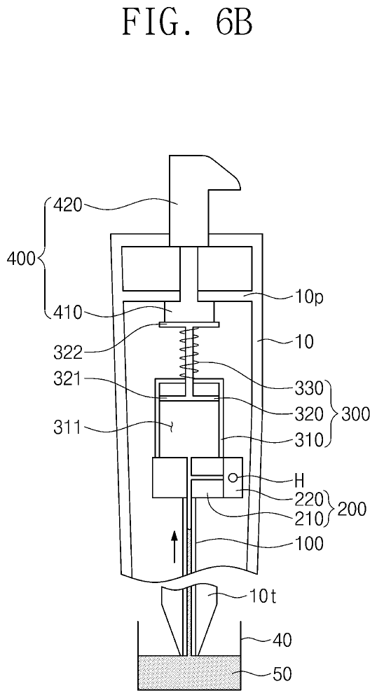

FIG. 5 is a flowchart illustrating a blood analysis method according to embodiments of the inventive concept. FIGS. 6A to 8, which are diagrams for describing a blood analysis method according to embodiments of the invention concept, are enlarged cross-sectional views of a part of a blood analysis apparatus.

Referring to FIGS. 5 and 6A, the cylinder 310 may be compressed by pressing the button part 400 (S10). An analysis target blood 50 may be provided in a specimen tub 40, and the tip portion 10t of the housing 10 may be dipped into the blood 50. The syringe part 300 may be operated by pressing the button part 400. Since movement of the upper button part 420 is restricted by the engaging protrusion 10p, the upper button part 420 may move the piston 320 downwards by a preset distance. As the piston 320 moves downwards, the volume of the internal space 311 of the cylinder 310 may reduce.

Referring to FIGS. 5 and 6B, the valve 220 of the pipe structure 200 may be closed (S20). In detail, the valve 220 may be closed so that the through-hole H of the valve 220 is not connected to the third opening OP3 (see FIG. 2) in the pipe body 210.

Referring to FIGS. 5 and 6B, the blood 50 may be provided in the capillary tube 100 by expanding the cylinder 310 (S30). The syringe part 300 may be operated by releasing pressure applied to the button part 400 of the blood analysis apparatus. The piston 320 may be pressed by the elastic member 330 and moved in a direction that reduces pressure in the cylinder 310. As the pressure in the cylinder 310 reduces, the blood 50 in the specimen tub 40 may move into the capillary tube 100. Since a movable range of the upper button part 410 is restricted by the engaging protrusion 10p, the upper button part 410 may move the piston 320 by a preset distance.

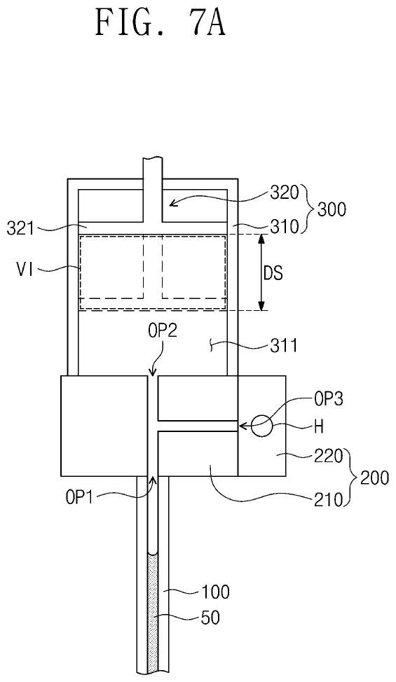

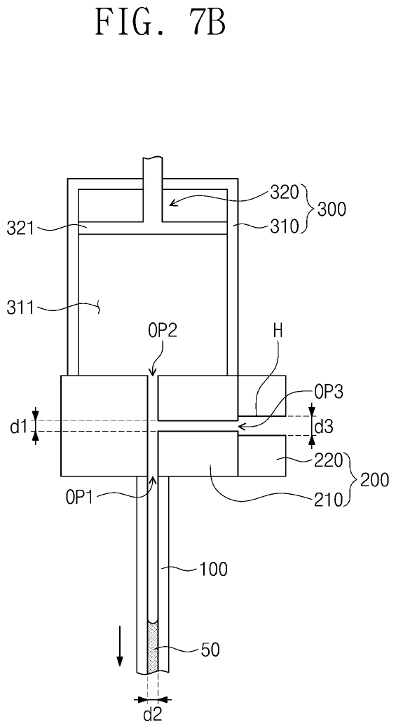

Referring to FIG. 7A, as the piston 320 moves, the volume of the internal space 311 of the cylinder 310 may increase. The blood 50 that is equivalent to an increased volume VI of the internal space of the cylinder 310 may be provided in the capillary tube 100. Referring to FIG. 7A together with FIG. 4, an amount of the blood 50 provided in the capillary tube 100 may be controlled by adjusting the length of the button part 400. For example, a movement distance DS of the piston 320 may be the same as the distance D between the lower surface of the upper button part 420 and the upper surface of the engaging protrusion 10p.

Referring to FIGS. 5 and 7B, the blood 50 may be discharged from the capillary tube 100 by opening the valve 220 of the pipe structure 200 (S40). In detail, the piston 320 may be fixed so that the volume of the internal space 311 of the cylinder 310 does not change. Thereafter, the third opening OP3 of the pipe body 210 may be opened by operating the valve 220. As the third opening OP3 is opened, pressure in the capillary tube 100 may be equalized to that of the outside of the capillary tube 100. The blood 50 provided in the capillary tube 100 may move downwards due to gravity.

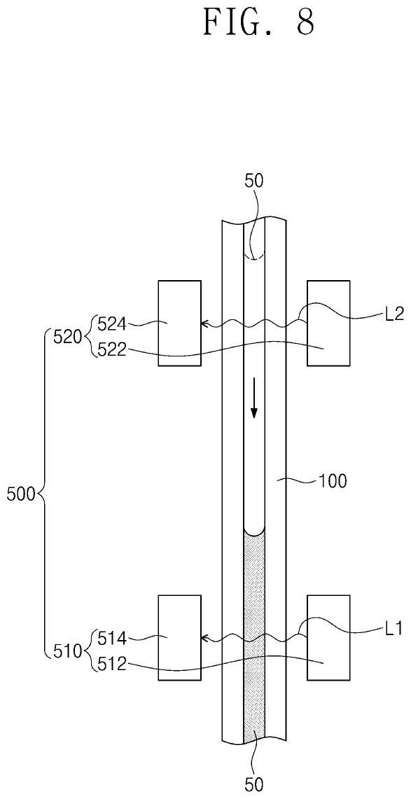

Referring to FIGS. 5 and 8, viscosity and hematocrit of the blood 50 moving in the capillary tube 100 may be measured using the photosensor unit 500 (S50). According to embodiments of the inventive concept, the blood 50 may sequentially pass through a sensing region of the second photosensor 520 and a sensing region of the first photosensor 510. The viscosity of the blood 50 may be measured by comparing a time at which the blood 50 passes through the sensing region of the second photosensor 520 with a time at which the blood 50 passes through the sensing region of the first photosensor 510. According to embodiments of the inventive concept, the blood analysis apparatus may further include a temperature sensor for measuring a temperature of the blood 50. The viscosity of the blood 50 may be more accurately measured using temperature information about the blood 50 and amount information about the blood 50 provided in the capillary tube 100. The amount of the blood 50 to be provided in the capillary tube 100 may be set in advance by controlling the button part 400. For example, the amount of the blood 50 provided in the capillary tube 100 may be calculated using the inner diameter of the cylinder 310 and an operation range of the button part 400. The amount of the blood 50 to be provided in the capillary tube 100 may be equally applied while analyzing a plurality of blood specimens. Therefore, the viscosity of the blood 50 may be measured reproducibly, and reliability of measurement of the viscosity of the blood 50 may be improved.

The hematocrit may be measured using the first photosensor 510 and the second photosensor 520. The hematocrit may represent a ratio of a volume of red blood cells to a volume of whole blood. First light L1 emitted from the first light transmission part 514 and second light L2 emitted from the second light transmission part 524 may have different wavelengths. For example, the first light L1 may have a wavelength of about 500 nm to about 600 nm. The second light L2 may have a wavelength of about 800 nm to about 1000 nm. The hematocrit of the blood 50 may be measured using the first light L1 input to the first light reception part 514 and the second light L2 input to the second light reception part 524. The hematocrit of the blood 50 may be calculated by comparing light absorption ratios of the blood 50 with respect to the first light L1 and the second light L2. The hematocrit may be corrected using the viscosity of the blood 50 and biomarker information in the blood 50. A method for correcting the hematocrit will be described in more detail.

According to embodiments of the inventive concept, a diameter d1 of the third opening OP3 of the pipe body 210 may be equal to an inner diameter d2 of the capillary tube 100. Accordingly, the blood 50 may be discharged from the capillary tube 100 at a fixed rate. The diameter d1 of the third opening OP3 may be, for example, about 0.1 mm to about 10 mm. A diameter d3 of the through-hole H of the valve 220 may be equal to or larger than the diameter d1 of the third opening OP3.

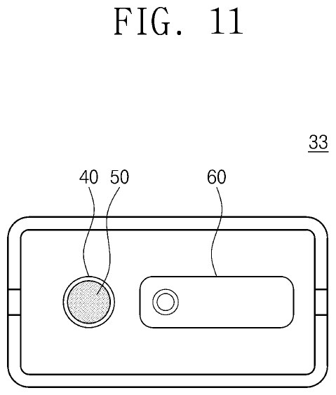

According to embodiments of the inventive concept, measuring the viscosity and hematocrit of the blood 50 (S50) may be performed simultaneously with detecting the biomarker in the blood 50 (S60) using the same blood specimen. According to embodiments of the inventive concept, the biomarker information in the blood 50 may be obtained by the absorption sensor 60 described below with reference to FIG. 11.

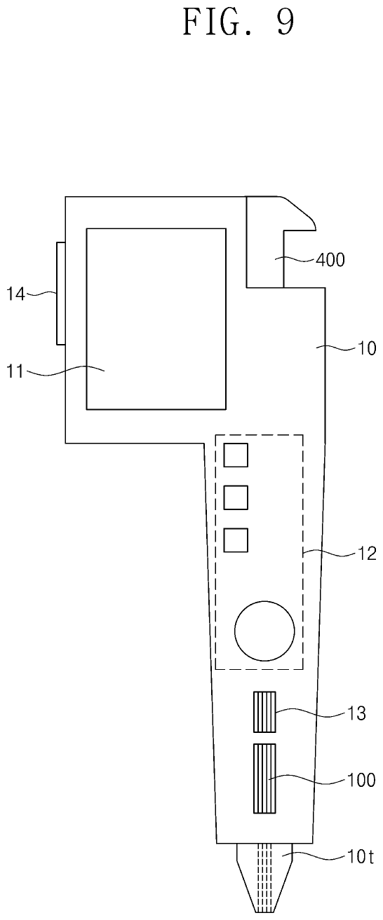

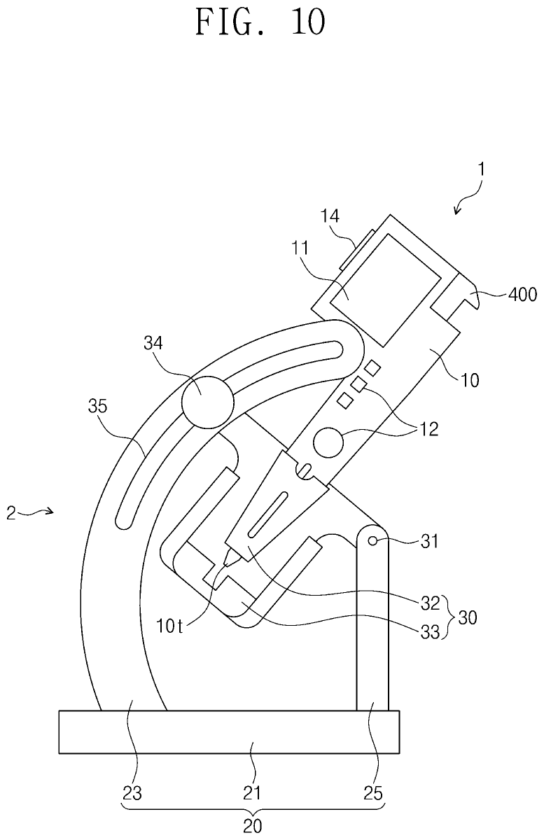

FIGS. 9 and 10 are front views of a blood analysis apparatus according to embodiments of the inventive concept.

Referring to FIGS. 1 and 9, the blood analysis apparatus may include a display part 11, an interface part 12, a window 13, and a connector part 14, which are provided on the housing 10.

The display part 11 may display blood analysis information and operation state information about the blood analysis apparatus. For example, the operation state information may include opening/closing information about the valve 220 and amount information about blood provided in the capillary tube 100. For example, the blood analysis information may include blood viscosity information, blood hematocrit information, and blood biomarker information measured by the photosensor part 500.

The interface part 12 may include a dial and buttons for operating the blood analysis apparatus. A user may input an opening/closing signal for the valve 220, an operation signal for the button 400, and the like through the interface unit 12.

The window 13 may be formed in a location corresponding to the capillary tube 100 so that the user may observe blood in the capillary tube 100.

The connector part 14 may include terminals for providing the blood analysis information to an external device.

Referring to FIGS. 9 and 10, the blood analysis apparatus may further include a cradle 2 supporting a measurement module 1. The cradle 2 may rotate the measurement module 1 so as to control an angle of the capillary tube 100 in the measurement module 1.

The cradle 2 may include a support part 20 and a holder part 30. The support part 20 may include a lower support stand 21, a first guide 23, and a second guide 25. The first guide 23 may have a slide hole 35. The slide hole 35 may have an arc shape with a pivot 31 of the second guide 25 as a center.

The holder part 30 may be rotatably coupled to the support part 20. The holder part 30 may be coupled to the first guide 23 through a slider 34 and may be coupled to the second guide 25 through the pivot 31.

The holder part 30 may include a module holder 32 and a lower sensor part 33. The module holder 32 may support the measurement module 1. The module holder 32 may have a basket part corresponding to a shape of a lower portion of the measurement module 1. The tip portion 10t of the measurement module 1 may protrude towards the lower sensor part 33.

The lower sensor part 33 may be provided to a lower portion of the holder part 30 and spaced apart from the tip portion 10t of the measurement module 1.

Referring to FIGS. 10 and 11, the lower sensor part 33 may include the specimen tub 40 and the absorption sensor 60. The lower sensor part 33 may be moved vertically and horizontally while fastened to the holder part 30. Accordingly, each portion of the lower sensor part 33 may be moved towards or away from the tip portion 10t of the measurement module 1.

The absorption sensor 60 may be configured to absorb the blood 50 and detect the biomarker in the blood 50. The absorption sensor 60 may include an immuno-sensing material that biologically reacts with immune substances in the blood 50. The immuno-sensing material may cause an immune reaction with the immune substances in the blood 50. The immune reaction may include at least one of various antigen-antibody immune reactions including C-reactive protein (CRP).

The analysis target blood 50 may be provided in the specimen tub 40. The specimen tub 40 may be located adjacent to the tip portion 10t of the measurement module 1 so as to provide the blood 50 in the measurement module 1. According to embodiments of the inventive concept, the lower sensor part 33 may not include the specimen tub 40. In this case, the measurement module 1 may be fastened to the cradle 2 while containing the analysis target blood 50.

Referring to FIGS. 5 and 8 to 11, the biomarker in the blood 50 discharged from the capillary tube 100 may be detected using the absorption sensor 60 (S60). Detecting the biomarker in the blood 50 (S60) may be performed simultaneously with measuring the viscosity and hematocrit of the blood 50 (S50) using the same blood specimen.

In detail, the absorption sensor 60 may be located under the tip portion 10t of the measurement module 1 during blood analysis so as to absorb the blood 50 discharged from the capillary tube 100. The absorption sensor 60 may detect the biomarker of the absorbed blood 50. The measurement module 1 may have pieces of characteristic information about the blood 50 discharged from the capillary tube 100. For example, the characteristic information about the blood 50 may include viscosity information and hematocrit information. By providing pieces of the characteristic information about the blood 50 to the absorption sensor 60, the measurement module 1 may improve accuracy of analysis of pieces of other characteristic information about the blood 50 measured by the absorption sensor 60.

Referring to FIGS. 1, 5, and 8 to 11, the hematocrit may be corrected using the viscosity and biomarker (S70). Correction of the hematocrit may be performed by the control part 15.

In detail, the control part 15 may measure and store the viscosity and biomarker information about the blood 50 on the basis of pieces of information input from the photosensor 500 and the absorption sensor 60. The control part 15 may use stored viscosity information and biomarker information about the blood 50 to correct a measurement value of the hematocrit according to the following equation.

HCT (corrected value)=HCT (measurement value)+m*Viscosity (measurement value)+n (where m and n are correction constants according to the type and amount of detected biomarker)

Here, the viscosity information and biomarker information used in correcting the hematocrit may be obtained from the same specimen as a specimen used in measuring the hematocrit.

Since blood is a heterogeneous mixture, even the same blood may exhibit different characteristics according to a portion thereof used as a specimen and elapse of time. Therefore, when the viscosity information and biomarker information obtained through a specimen that is different from a specimen used in measuring the hematocrit is used in correcting the hematocrit, reliability of the correction may deteriorate. The blood analysis apparatus according to embodiments of the inventive concept may simultaneously obtain various pieces of characteristic information with respect to one blood specimen. Therefore, a degree of precision of correction of a measurement value and blood analysis may be improved.

The blood analysis apparatus according to embodiments of the inventive concept may diagnose blood with ease using a syringe part and an opening/closing operation of a valve of a pipe structure connected to the syringe part. For example, a fixed and small amount of blood may be directly provided in a capillary tube by operating the syringe part after closing the valve. Thereafter, the valve is opened, and thus the above-mentioned variety of blood characteristic information may be measured highly precisely. Therefore, the blood analysis apparatus according to embodiments of the inventive concept may enable a user to perform a self-blood diagnosis with ease using only a small amount of blood.

Various pieces of blood characteristic information may correlate with each other, and may exhibit particular tendencies with regard to particular diseases. Since the blood analysis apparatus according to embodiments of the inventive concept is capable of simultaneously obtaining various pieces of characteristic information with respect to a small amount of blood specimen, the accuracy of disease diagnosis based on blood analysis may be improved. That is, the blood analysis apparatus according to embodiments of the inventive concept may allow a user to conveniently perform a self-diagnosis of disease.

According to embodiments of the inventive concept, a blood analysis apparatus with improved precision, reproducibility, and reliability of measurement of the viscosity and hematocrit of blood may be provided. Furthermore, the blood analysis apparatus according to embodiments of the inventive concept may simultaneously obtain various pieces of characteristic information with respect to one blood specimen. Therefore, deterioration of analysis precision caused by a difference between blood specimens may be prevented, and the accuracy of disease diagnosis based on blood analysis may be improved.

Although the embodiments of the present invention have been described, it is understood that the present invention should not be limited to these embodiments but various changes and modifications can be made by one ordinary skilled in the art within the spirit and scope of the present invention as hereinafter claimed.

What is claimed is:

1. A blood analysis apparatus comprising:
a capillary tube;
a photosensor part on a sidewall of the capillary tube;
a cylinder connected to the capillary tube;
a pipe body connecting the cylinder and the capillary tube and comprising a first opening connected to an internal space of the cylinder, a second opening connected to an internal passage of the capillary tube, and a third opening connected to the first opening and the second opening; and
a valve arranged on the pipe body and configured to open/close the third opening.

2. The blood analysis apparatus of claim 1, wherein the third opening has a diameter equal to an inner diameter of the capillary tube.

3. The blood analysis apparatus of claim 1, wherein the first opening and the second opening vertically overlap the internal passage of the capillary tube, and the third opening is horizontally spaced apart from the internal passage of the capillary tube.

4. The blood analysis apparatus of claim 1, wherein the third opening has a diameter of about 0.1 mm to about 10 mm.

5. The blood analysis apparatus of claim 1, wherein the valve comprises a through-hole aligned with the third opening, and is rotatably coupled to the pipe body.

6. The blood analysis apparatus of claim 1, wherein the valve comprises a through-hole aligned with the third opening, wherein a diameter of the through-hole is equal to or larger than a diameter of the third opening.

7. The blood analysis apparatus of claim 1,
wherein the photosensor part comprises a first photosensor and a second photosensor between the first photosensor and the pipe body,
wherein the second photosensor is configured to transmit and receive light of a wavelength that is longer than that of light transmitted and received by the first photosensor.

8. The blood analysis apparatus of claim 1, further comprising a piston coupled to the cylinder,
wherein the piston comprises a first end portion in the cylinder, a second end portion outside the cylinder, and a connecting part penetrating one end of the cylinder and connecting the first end portion and the second end portion.

9. The blood analysis apparatus of claim 8, further comprising an elastic member between the second end portion and the one end of the cylinder.

10. The blood analysis apparatus of claim 1, further comprising a piston coupled to one end of the cylinder and a button part on the piston,
wherein the button part comprises an upper button part and a lower button part coupled to the upper button part, and
wherein the button part is configured to adjust a maximum movable range of the piston by adjusting a distance between a lower surface of the lower button part and a lower surface of the upper button part.

11. The blood analysis apparatus of claim 1, wherein a maximum volume of the internal space of the cylinder is larger than a volume of the internal passage of the capillary tube.

12. The blood analysis apparatus of claim 1, wherein the first opening has a diameter equal to an inner diameter of the capillary tube and is aligned with the internal passage of the capillary tube.

13. A blood analysis apparatus comprising:
a capillary tube;
photosensors on a sidewall of the capillary tube;
a pipe structure on one end of the capillary tube;
a syringe part on the pipe structure; and
a button part on the syringe part,
wherein the syringe part comprises a cylinder connected to the capillary tube through the pipe structure and a piston coupled to the cylinder,
wherein the piston comprises a first end portion in the cylinder, a second end portion outside the cylinder, and a connecting part penetrating one end of the cylinder and connecting the first end portion and the second end portion, and
wherein the button part is arranged on the second end portion.

14. The blood analysis apparatus of claim 13, further comprising an elastic member between the one end of the cylinder and the second end portion.

15. The blood analysis apparatus of claim 13, further comprising a sealing member between the connecting part and the cylinder.

16. The blood analysis apparatus of claim 13, wherein the pipe structure comprises a pipe body including an opening connected to the cylinder and the capillary tube and a valve arranged on the pipe body and configured to open/close the opening.

17. The blood analysis apparatus of claim 16, wherein the opening has a diameter equal to an inner diameter of the capillary tube.

18. The blood analysis apparatus of claim 13, further comprising an absorption sensor arranged adjacent to the one end of the capillary tube.

19. The blood analysis apparatus of claim 13,
wherein the button part comprises an upper button part and a lower button part inserted into the upper button part to a first depth, and
wherein the button part is configured to control a maximum movable range of the piston by adjusting the first depth.

20. The blood analysis apparatus of claim 13, further comprising a control part configured to correct hematocrit using viscosity information and biomarker information about blood.
